(19) United States
(12) Patent Application Publication
(10) Pub. No.: US 2008/0032391 A1
(43) Pub. Date: Feb. 7, 2008

(54) INSPECTION APPARATUS

(75) Inventors: Muneki ARARAGI, Tokyo (JP); Takeo Tanaami, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: YOKOGAWA ELECTRIC CORPORATION, Tokyo (JP)

(21) Appl. No.: 11/832,761

(22) Filed: Aug. 2, 2007

(30) Foreign Application Priority Data
Aug. 3, 2006 (JP) 2006-212195

(51) Int. Cl. C12M 1/34

(52) U.S. Cl. 435/288.2

(57) ABSTRACT

Disclosed is an inspection apparatus that includes a cartridge including a container which is at least partially structured with an elastic member, the container including inside thereof a plurality of rooms to contain solution and a passage to connect the plurality of rooms; and a housing which is arranged with a liquid feeding device which conducts inspection of the solution by applying external force to the elastic member to move the solution in the passage or in at least one of the rooms, wherein the housing is sealed air-tightly from outside air, and the housing is a safety cabinet to which a sterilization filter is provided at an outlet passage thereby that communicates from inside to external of the housing.

### BACKGROUND OF THE INVENTION

1. Field of the Invention

The present invention relates to an inspection apparatus, which is capable of conducting inspection such as mixing, synthesizing, dissolving, separating and the like, automatically in a state where a pathogen which is concerned to be a source of infection and hazardous inspection samples are completely blocked.

2. Description of the Related Art

In recent years, in a case where a pathogen is handled in an inspection room or the like, importance is placed on measures against biohazard to prevent a worker in the inspection room from being infected by the pathogen such as bacteria and virus, and to avoid leakage of the pathogen. The foremost measure with respect to prevention of infection in the inspection room is to provide training of practical skill to handle the pathogen. However, no matter how highly the worker is skilled in handling the pathogen, aerosol infection, splash infection and the like cannot be prevented without special care and facilities. Therefore, recognition of bio-safety level of the pathogen, and prevention measures with respect to each of the levels are required.

Accordingly, in a case where the bio-safety level is such that hazardous degree of infection and the like is mild or moderate, it is required to conduct inspection in an inspection room provided with a safety cabinet. The safety cabinet is designed so as to contain hazardous microorganism and to avoid leakage of the microorganism to the outside of the cabinet. For example, a safety cabinet in which the worker can insert his or her upper-body or hands into a working space provided with an inlet and an outlet, and conduct operation, has been known (refer to Japanese Patent Application Publication (laid open) Nos. 2000-279161 and 2005-235882).

However, with respect to the afore-mentioned safety cabinet, operation is conducted by inserting the upper-body or the hands of the worker. Therefore, glass apparatus, processing device, measuring/inspection device and the like that are necessary for the worker to conduct inspection were arranged in the safety cabinet, thus resulted in a relatively large apparatus. Further, since such apparatus is furnished in a region where it is managed (controlled) in accordance with bio-safety levels, there is a problem that inspection without time lapse, such as in a case where inspection is conducted on the scene where a specimen is collected from a patient, cannot be managed.

The present invention has been made in light of the afore-mentioned circumstances. An object of the present invention is to provide an inspection apparatus which enables inspection in a state where a pathogen which is concerned to be a source of infection and hazardous inspection samples are completely blocked, and further enables rapid and simple processing by automation of the inspection and downsizing of the apparatus.

### SUMMARY OF THE INVENTION

According to a first aspect of the present invention, there is provided an inspection apparatus, comprising:

a cartridge including a container which is at least partially structured with an elastic member, the container including inside thereof a plurality of rooms to contain solution and a passage to connect the plurality of rooms; and

a housing which is arranged with a liquid feeding device which conducts inspection of the solution by applying external force to the elastic member to move the solution in the passage or in at least one of the rooms, wherein the housing is sealed air-tightly from outside air, and the housing is a safety cabinet to which a sterilization filter is provided at an outlet passage thereby that communicates from inside to external of the housing.

### BRIEF DESCRIPTION OF THE DRAWINGS

The above and further objects, features and advantages of the present invention will become more apparent from the following detailed description taken in conjunction with the accompanying drawings, wherein:

FIG. 1 is a schematic perspective view showing an inspection apparatus 100 according to the present invention;

FIG. 2 is a partially cutaway view showing the inspection apparatus 100 of FIG. 1;

FIG. 3 is a perspective view of the inspection apparatus 100, precisely showing a liquid feeding device 4 and the like that are included in a housing 5;

FIG. 4 is a plan sectional view of FIG. 3;

FIG. 5A is a perspective view of a cartridge 3;

FIG. 5B is a top view of the cartridge 3;

FIG. 5C is a view showing a sectional view of the cartridge 3 taken along a cutting line of V-V when seen in the direction of the arrow in FIG. 5C;

FIG. 6A is a plan view showing an operation of a squeegee 41;

FIG. 6B is a plan view showing the operation of the squeegee 41;

FIG. 6C is a plan view showing the operation of the squeegee 41;

FIG. 7 is a perspective view of the inspection apparatus 100A, precisely showing a liquid feeding device 4A and the like that are included in a housing 5A; and

FIG. 8 is a plan sectional view of FIG. 7.

### DESCRIPTION OF THE PREFERRED EMBODIMENTS

Hereinafter, embodiments according to the present invention will be described with reference to the drawings.

### First Embodiment

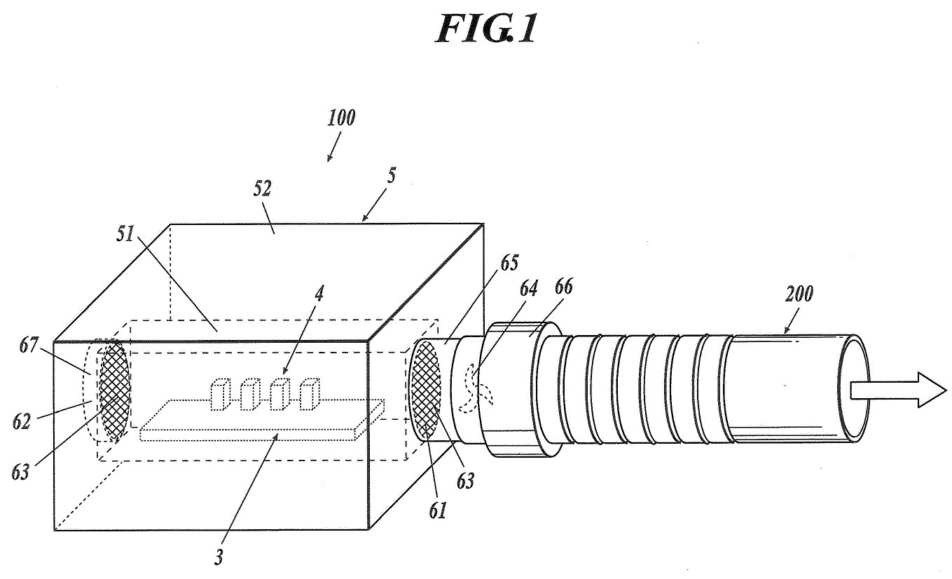

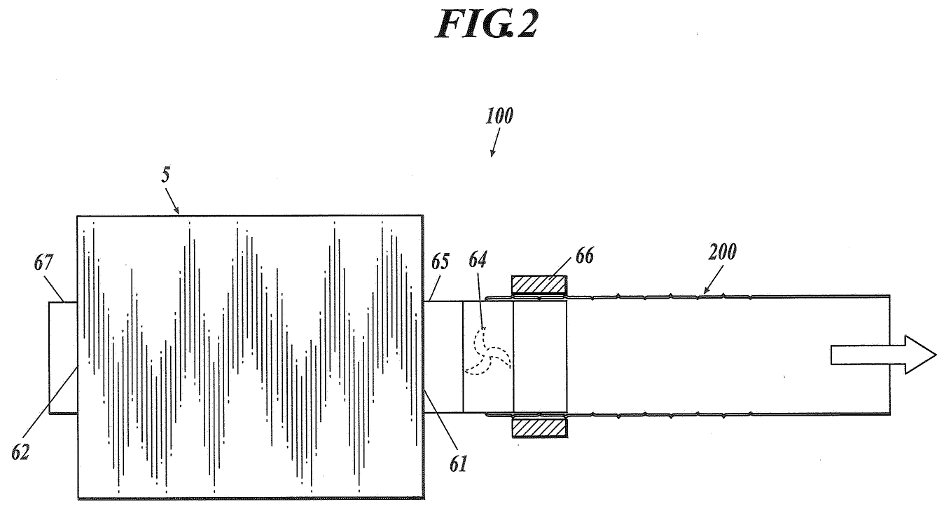

FIG. 1 is a schematic perspective view showing an inspection apparatus 100, and FIG. 2 is a partially cutaway view showing the inspection apparatus 100 of FIG. 1.

The inspection apparatus 100 is provided with a housing 5, which is arranged with a cartridge 3 charged with a solution as an inspection sample, and with a liquid feeding device 4 to conduct inspection of the solution by applying external force to the cartridge 3 and by feeding the solution into the cartridge 3.

The housing 5 is box-shaped, and serves as a safety cabinet which is sealed from the outer atmosphere. At the right side of the housing 5, an outlet 61 is provided to emit discharge air in the housing 5 (processing area 51 described later) to the outside. A discharge duct 65 is connected to the outlet 61, and a sterilization filter 63 is attached to the discharge duct 65. At the left side of the housing 5, an inlet 62 is provided to take outside air into the housing 5 (processing area 51). A suction duct 67 is connected to the inlet 62, and a sterilization filter 63 is attached to the suction duct 67. The inside of the housing 5 is aspirated so as to have negative pressure, and thus air in the housing 5 does not leak to the outside naturally. As for the sterilization filter 63, High Efficiency Particulate Air (HEPA) filter can be used for example. The housing 5 is structured so that the cartridge 3 can be inserted in the processing area 51 in the housing 5 by opening and closing an opening which is sealed to the inlet 62, for example.

At the outside of the sterilization filter 63 of the outlet 61 side, a fan 64 is provided so as to efficiently discharge the air inside the housing 5 to the outlet 61. Further, a connecting unit (connecting section) 66 is provided at the outside of the fan 64, the connecting unit connecting to an external processing device 200. The external processing device 200 is a device to send the discharge air to the outside of the room for example, and the discharge air sent through the connecting unit 66 is emitted to the outside of the room by the external processing device 200.

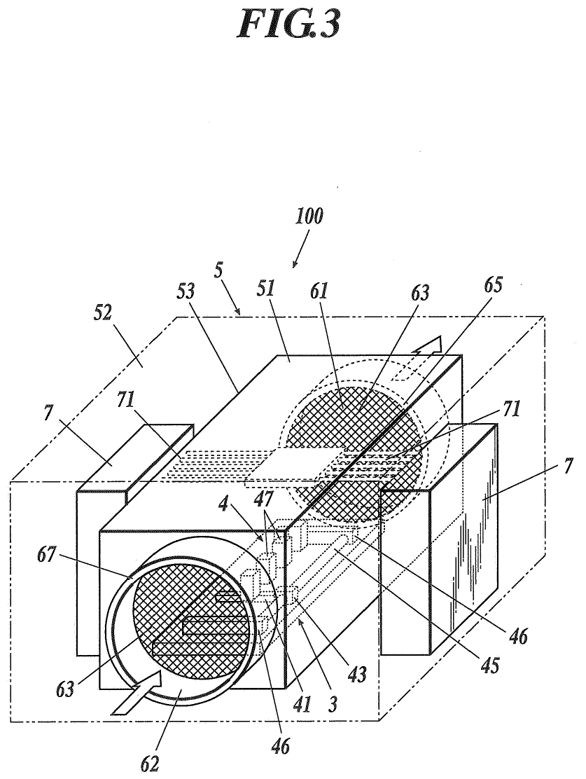

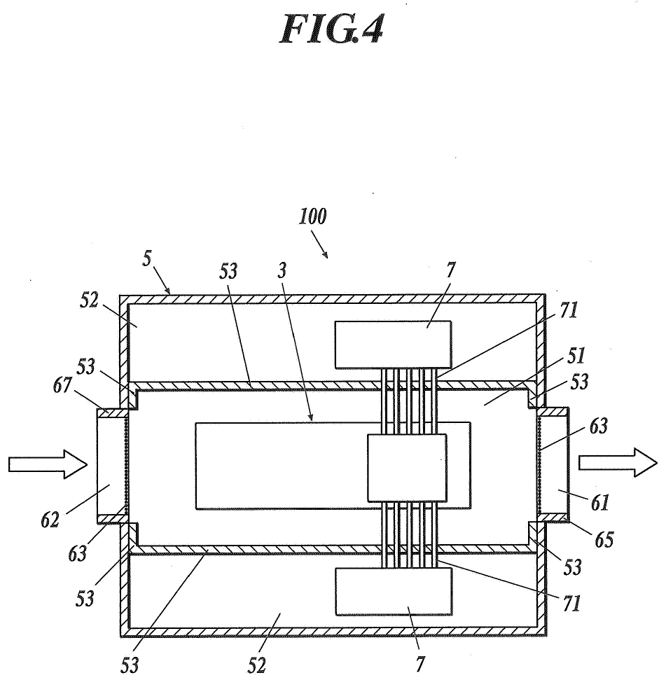

FIG. 3 is a perspective view of the inspection apparatus 100, precisely showing a liquid feeding device 4, driving source 7 and the like that are included in the housing 5; and FIG. 4 is a plan sectional view of FIG. 3. Here, for convenience, a squeegee 41, a slider 43, a guide rail 45 and the like are omitted in FIG. 4.

In the housing 5, the cartridge 3 and the liquid feeding device 4 are arranged. Further, the inside of the housing is provided with partition walls 53, 53, . . . to partition the processing area 51 which conducts inspection by the cartridge 3, from a containing area which contains an electric circuit and a mechanical device that serve as driving sources 7, 7 of the liquid feeding device 4, air piping, and the like. The partition walls 53, 53, . . . are provided at each of the top, bottom, left, right, front and back side of the housing 5, making the housing 5 have a double structure, and thus two spacial portions are formed. The internal spacial portion which is formed by six partition walls 53, 53, . . . is the processing area 51, and the spacial portion which is formed at the outside of the processing area 51 is the containing area 52. In addition, with respect to the processing area 51, part of the left and right partition walls 53, 53 respectively communicates with outlet 61 and the inlet 62, and outside air is taken in from the inlet 62 and emitted to the outlet 61. The containing area 52 does not communicate with the outlet 61 and the inlet 62, and is isolated by the left and right partition walls 53, 53.

In the processing area 51, the cartridge 3 is set, and the liquid feeding device 4 is arranged above the cartridge 3. With respect to the containing area 52, in the front surface side and the back surface side, the electric circuit and the mechanical device that serve as driving sources 7, 7 of the liquid feeding device 4, air piping, and the like are each contained. Further, wiring (transfer section) 71, 71 that electrically connect the liquid feeding device 4 and its driving source 7, 7 such as the electric circuit, are arranged in the processing area 51 by allowing the wiring penetrate through the front and back partition walls 53, 53. Penetrating portion of the wiring 71, 71 and the partition walls 53, 53 are sealed air-tight. In this way, to the processing area 51, only the cartridge 3, the liquid feeding device 4, and the wiring 71, 71 that are sealed air-tight, are exposed. Since the electric circuit and the mechanical device, an air pressure providing device, an oil pressure providing device and the like that serve as the driving source 7, 7 are not exposed to the processing area 51, they are not polluted by the pathogen and the like. Further, internal surface of each of the partition walls 53, 53, . . . are insulated and are sealed air-tight by the air-tight seal.

Here, although not shown, in a case where the driving sources 7, 7 are the mechanical device, an axis (transfer section) and the like to transfer mechanical force is arranged in the processing area 51 by allowing the axis to penetrate through the partition walls 53, 53, and the axis and the like are sealed air-tight. Further, in a case where the driving sources 7, 7 are the air pressure providing device or the oil pressure providing device, piping (transfer section) or wiring (transfer section) to send air pressure or oil pressure is arranged in the processing area 51 by allowing the piping or the wiring to penetrate through the partition walls 53, 53, and these piping or the wiring is sealed air-tight. A structure in which the transfer section is an electric wiring and actuators or driving source is provided in the processing area 51 can also be used as a matter of course.

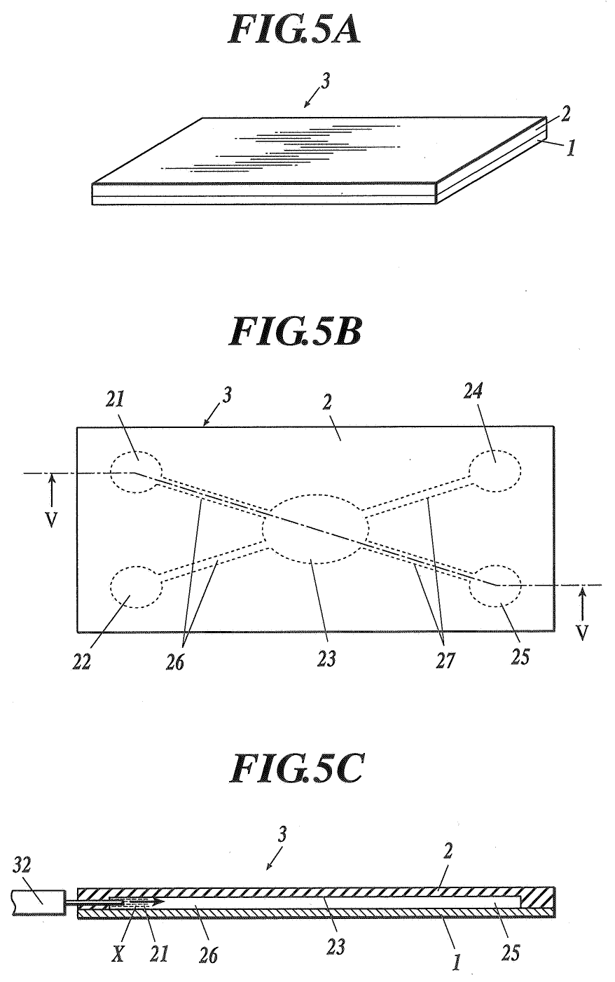

FIG. 5A is a perspective view of a cartridge 3; FIG. 5B is a top view of the cartridge 3; and FIG. 5C is a view showing a sectional view of the cartridge 3 taken along a cutting line of V-V when seen in the direction of the arrow in FIG. 5C.

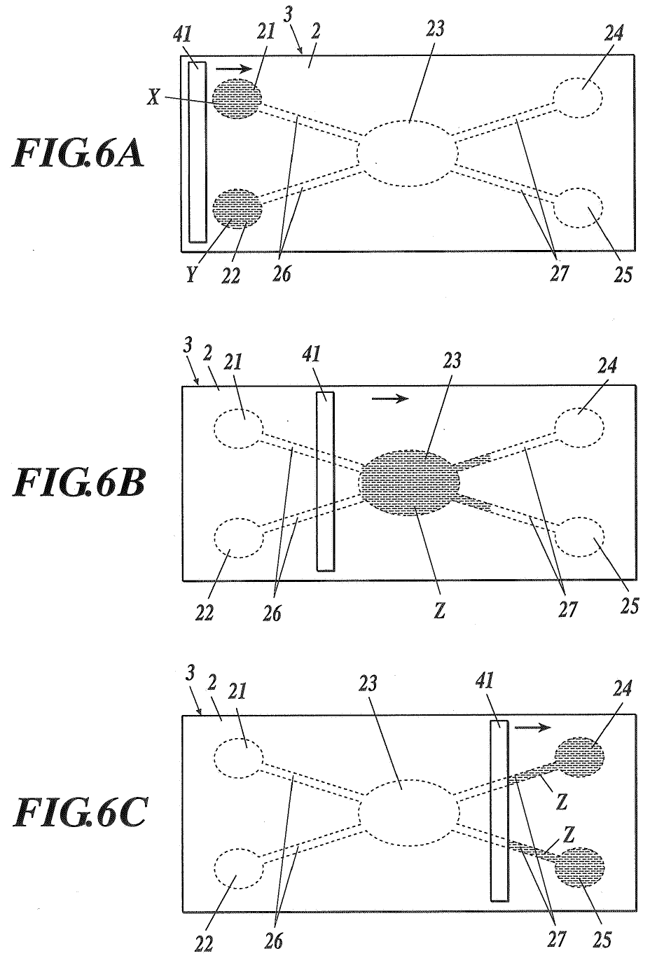

The cartridge 3 is formed by overlapping an elastic member 2 on a substrate 1. In between the substrate 1 and the elastic member 2, a plurality of rooms 21-25 that contain solutions X, Y and Z (refer to FIG. 6) and passages 26, 26, 27 and 27 that connect the rooms 21-25 with each other are formed. Here, a container is structured by the substrate 1 and the elastic member 2.

The substrate 1 is made of a hard material, and is long and flat-shaped so as to specify location and to maintain its shape.

The elastic member 2 is made of a silicone rubber such as polydimethylsiloxane (PDMS) or a polymer material which has air-tight property and has elasticity. The elastic member 2 has a size similar to that of the substrate 1, and is long and flat-shaped. Here, as for the elastic member 2, a viscoelastic member or a plastic member can be used other than rubber. To the bottom surface of the elastic member 2 which contacts with the surface of the substrate 1, a plurality of concave portions for solution, each of which being able to concave in an upper direction so as to expand, are formed. These plurality of concave portions serve as injection rooms 21 and 22 for injection in which the solution is injected, a reaction room 23 for reaction unit in which the solution in the injection rooms 21 and 22 react, and divisionally ejecting rooms 24 and 25 for divisional injection into which the solution that reacted in the reaction room 23 is divisionally injected. Further, to the bottom surface of the elastic member 2, passages 26 and 26 that connect each of the injecting rooms 21 and 22 to the reaction room 23, and passages 27 and 27 that connect each of the divisionally ejecting rooms 24 and 25 to the reaction room 23, are formed. The injecting rooms 21 and 22 and the divisionally ejecting rooms 24 and 25 have circular form when observed from top, and the reaction room 23 has ellipse form when observed from top. A bonding region, which excludes the injecting rooms 21 and 22, the reaction room 23, the divisionally ejecting rooms 24 and 25, and the passages 26, 26, 27 and 27, in the bottom surface of the elastic member 2 is bonded to the top surface of the substrate 1. Accordingly, the injecting rooms 21 and 22, the reaction room 23, the divisionally ejecting rooms 24 and 25, and the passages 26, 26, 27 and 27 are sealed air-tight by the elastic member 2 and the substrate 1, and thus forms a later-described structure in which leakage of solutions X, Y and Z to the outside are prevented.

As shown in FIGS. 3 and 6, the liquid feeding device 4 is provided with a squeegee 41, an actuator such as a guide rail 45, a slider 43 and the like that moves the squeegee 41 in a longitudinal direction of the cartridge 3, a driving source 7 to drive the actuator, and the like. Here for example, the squeegee 41 applies external force to the elastic member 2 by moving while being in contact with the top surface of the elastic member 2. Accordingly, the passages 26, 26, 27, 27 and/or the rooms 21-25 are partly blocked, and thus solutions X and Y in the blocked passages 26, 26, 27, 27 or the rooms 21-25 are moved.

The squeegee 41 for example, is long and has a prismatic shape, and is elongated along a shortitudinal direction of the cartridge 3. The squeegee 41 moves independently while being in contact with the top surface of the elastic member 2.

The guide rail 45 is elongated in the longitudinal direction of the cartridge 3, and is supported onto the end portions of the top surface of the cartridge 3 by supporting members 46 and 46 that are provided along in the shortitudinal direction of the cartridge 3. Further, the slider 43 is provided to the guide rail 45, so that the slider 43 is capable of moving independently along the guide rail 45. The squeegee 41 is fixed to the slider 43, so that the squeegee 41 is elongated in the shortitudinal direction of the cartridge 3. Accordingly, when the slider moves along the guide rail 45, the squeegee 41 also moves along the guide rail 45 and the top surface of the cartridge is depressed, allowing the feeding of the solutions.

As for the driving sources 7 and 7 of the actuator, electric power, mechanic power, air pressure, or oil pressure can be mentioned. In the drawings, reference numeral 47 shows a measure to connect driving force from the driving source 7 (electric power, mechanic power, air pressure, oil pressure and the like) with the slider 43 and the like.

Next, liquid feeding operation will be described. FIGS. 6A-6C are plan views showing an operation of the squeegee 41.

First of all, solution X and solution Y are each injected beforehand, to the injecting rooms 21 and 22 that are formed in the cartridge 3. Injection into the injecting rooms 21 and 22 is conducted by, for example as shown in FIG. 5C, direct injection to the elastic member 2 using a syringe 32.

FIG. 6A shows a state after the injection of the solutions X and Y, and before the feeding of the solutions. The squeegee 41 is located at the top surface in the left end portion of the elastic member 2, and the bottom surface of the squeegee 41 is in contact with the top surface of the elastic member 2 so as to depress the elastic member 2. Starting from this state, the movement of the slider 43 from left side to right side along the guide rail 45 allows the movement of the squeegee 41 to the right side along the top surface of the elastic member 2. During this movement, the top surface of the elastic member 2 is depressed by the bottom surface of the squeegee 41, pressing out the solutions X and Y contained in the injecting rooms 21 and 22 to move into the reaction room 23 through the passages 26 and 26.

As shown in FIG. 6B, the squeegee 41 further moves to right side along the top surface of the elastic member 2, by the slider 43. Also during this movement, the top surface of the elastic member 2 is depressed by the bottom surface of the squeegee 41, and the solution contained in the passages 26 and 26, and in the reaction room 23 is pressed out in the right direction. Thus, when the squeegee 41 moves over the reaction room 23, the solutions X and Y that are fed to the reaction room 23 are mixed and reacted. Here, reaction refers to mixing, synthesizing, dissolving, separating and the like. By using the cartridge 3 as described above, detection of dioxin, DNA and the like can be conducted. Further, since the squeegee 41 depresses the top surface of the elastic member 2, back flow of the fed solution can be prevented.

The solution Z which is the solution after being reacted in the reaction room 23, is then moved from the passages 27 and 27 to the divisionally ejecting rooms 24 and 25, by the movement of the squeegee 41 as shown in FIG. 6C.

As described above, the inspection apparatus 100 is provided with the housing 5 which is arranged with the cartridge 3 and the liquid feeding device 4 which automatically conducts inspection by moving the solutions X and Y in the cartridge 3. Further, the housing 5 is sealed air-tight from the outside air, and sterilization filter 63 is provided to the outlet 61. Therefore, a series of processing related to inspection such as mixing, synthesizing, dissolving, separating, and detecting, can be conducted in a state where source of infection such as a pathogen and hazardous inspection samples are completely blocked. In addition, since the inspection of the solutions X and Y can be automated, workers need not to conduct the operation and thus workers are prevented from infection. Further, since the workers need not to use apparatuses and the like that are necessary for the inspection in the housing 5, re-infection by the polluted apparatuses and the like can be prevented, and the apparatus 100 can be downsized. In particular, the cartridge 3 is a card-like form with a palm-size, the liquid feeding device 4 can be downsized to a desk-top size, and thus the housing 5 which is arranged with the cartridge 3 and the liquid feeding device 4 can also be downsized to a desk-top size. Since the inspection is conducted automatically, inspection result will not be effected by a working efficiency of individuals, and thus reproducibility is high.

By realization of downsizing as described above, at a site away from a conventional inspection room or laboratory, inspection can be conducted immediately in a case where inspection is required. In addition, since the apparatus is not as large as the conventional apparatus, there is no limitation with respect to the space of the site, and thus inspection can be conducted easily.

The present invention has been described with presumption for an inspection of a biological sample such as infected body or a blood, which requires handling set for bio-safety level under 2. However, the present invention is not limited to such level, and may be used for other levels so long as it is provided with necessary functions in a controlled region.

In the housing 5, partition walls 53, 53, . . . that partition the processing area 51 form the containing area 52 are provided, and the liquid feeding device 4 and the driving source 7 are connected by the wirings 71 and 71 that penetrates the partition walls 53, 53, . . . while the penetrating portion of the wirings 71 and 71 that are exposed to the processing area 51 are sealed air-tight. Accordingly, the driving sources 7 and 7 are not polluted and only the liquid feeding device 4 and the cartridge 3 are arranged in the processing area 51, since they are partitioned by the partition walls 53, 53, . . . Therefore, only the cartridge 3 and the liquid feeding device 4 are polluted. From this point also, re-infection from polluted apparatuses due to pollution of the apparatuses and the like can be prevented.

Further, since the connecting unit 66, which is connected to the external processing device 200, is provided to the discharge duct 65, emission to the external processing device 200 can easily be conducted through the connecting unit 66, and emission processing can be conducted securely.

### Second Embodiment

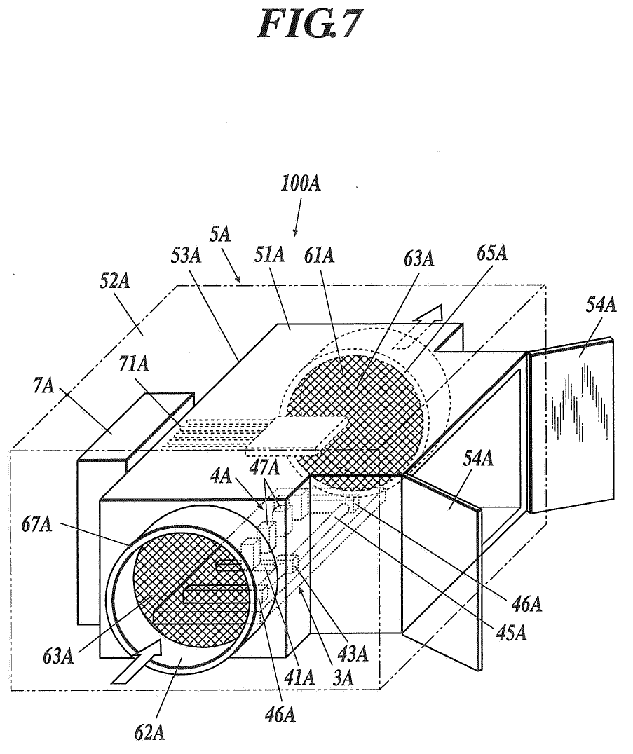

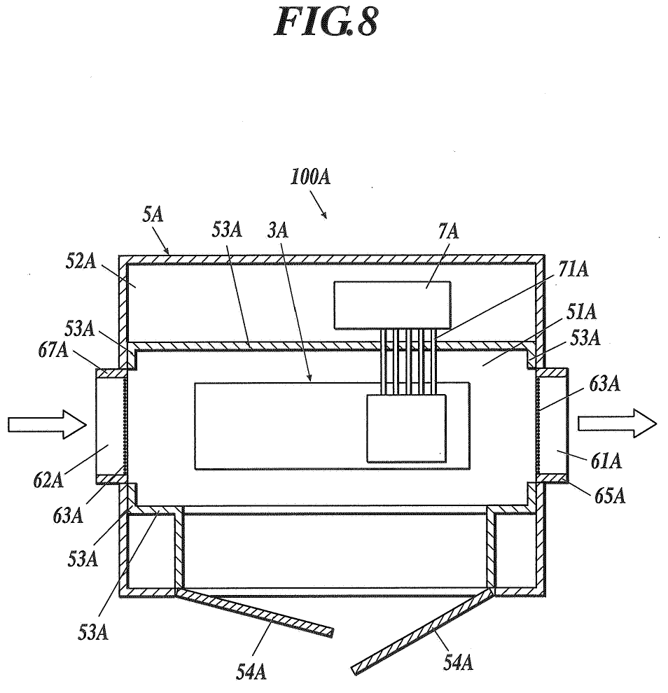

FIG. 7 is a perspective view of the inspection apparatus 10A, precisely showing a liquid feeding device 4A and the like that are included in a housing 5A; and FIG. 8 is a plan sectional view of FIG. 7. Here, for reasons of drawings, a squeegee 41A, a slider 43A, a guide rail 45A and the like are omitted in FIG. 7.

In contrast to the inspection apparatus 100 according to the first embodiment, the inspection apparatus 100A according to the second embodiment is provided with cleaning door 54A, in the front of the housing 5A so as to communicate with the processing area 51A. The driving source 7A is provided only in the containing area 52A which is in the back side of the housing 5A. Since other structures are the same as the first embodiment, the same structuring portions are provided with the same numeral with alphabet A, and description thereof is omitted.

As shown in FIGS. 7 and 8, the cleaning door 54A is provided in a gate fold manner, and is formed so as to penetrate through the partition wall 53A formed on the front surface of the housing 5A, and to communicate with processing area 51A. By opening the cleaning door 54A, cleaning of the processing area 51A can be conducted. Here, since inside the processing area 51A has negative pressure, air does not leak to the outside. In addition, the cartridge 3A can be inserted into the processing area 51A through the cleaning door 54A.

Further, internal surface of the partition wall 53 and a portion of the cleaning door 54A that face the processing area 51A are insulated and are sealed air-tight by the air-tight seal.

Here, the present invention is not limited to the afore-mentioned embodiments, and modifications can be made so long as it does not deviate the scope of the invention.

For example, in the afore-mentioned embodiment, air-tight seal may not be limited to glass sealing, and sealing by a rubber packing or an O-ring may also be used so long as it can maintain air-tight.

In addition, sterilization filters 63, 63 and 63A, 63A were provided each of the outlets 61 and 61A, and inlets 62 and 62A by one respectively. However, they may be provided in plural. Further, outlets 61 and 61A, and inlets 62 and 62A are each not limited to one, and may be provided in plural at other portions of the housings 5 and 5A.

With respect to the liquid feeding device 4 and 4A, squeegees 41 and 41A were used in the afore-mentioned embodiments, however, rollers may be used as well. Further, number of the squeegees or the rollers may be altered arbitrarily, and thus number of the sliders 43 and 43A are also altered accordingly. Location and number of the cleaning door 54A, as well as location and size of the processing areas 51 and 51A, and containing areas 52 and 52A are also not limited to the ones described above. Further, shape, number and the like of the plurality of rooms 21-25 and passages 26, 26, 27, 27 can also be altered.

Further, with respect to the elastic member 2, the elastic member is required to be arranged to at least a portion that corresponds to the rooms and passages.

According to the present invention, there is provided an inspection apparatus, comprising:

a cartridge including a container which is at least partially structured with an elastic member, the container including inside thereof a plurality of rooms to contain solution and a passage to connect the plurality of rooms; and

a housing which is arranged with a liquid feeding device which conducts inspection of the solution by applying external force to the elastic member to move the solution in the passage or in at least one of the rooms,

wherein the housing is sealed air-tightly from outside air, and the housing is a safety cabinet to which a sterilization filter is provided at an outlet passage thereby that communicates from inside to external of the housing.

Preferably, the cartridge and the liquid feeding device are arranged inside the housing,

a partition wall is provided inside the housing, the partition wall partitioning a processing area in which the inspection of the solution is conduced by applying external force to the elastic member, from a containing area in which a driving source to drive the liquid feeding device is accommodated,

the liquid feeding device and the driving source are connected with each other by a transfer section which penetrates through the partition wall, and

the transfer section transfers a driving force from the driving source to the liquid feeding device, while the transfer section is sealed air-tight with respect to the partition wall.

Preferably, the housing is provided with a cleaning door to conduct cleaning of inside the processing area.

Preferably, the inspection apparatus further comprises:

a discharge duct to emit discharge air from inside the housing;

wherein the discharge duct is provided with a connecting section that connects with an external processing device.

According to the present invention, since the housing is sealed air-tight from the outside air, and the housing is a safety cabinet in which the sterilization filter is provided to the outlet passage that communicates from the inside of the housing to the external, inspection can be conducted in a state where source of infection such as a pathogen and hazardous inspection samples are completely blocked. In addition, since the liquid feeding device conduct the inspection automatically, inspection is automated and the apparatus can be downsized. Accordingly, inspection can be conducted rapidly and easily.

The entire disclosure of Japanese Patent Application No. 2006-212195 filed on Aug. 3, 2006 including specification, claims, drawings and abstract are incorporated herein by reference in its entirety.

Although various exemplary embodiments have been shown and described, the invention is not limited to the embodiments shown. Therefore, the scope of the invention is intended to be limited solely by the scope of the claims that follow.

1. An inspection apparatus, comprising:
a cartridge including a container which is at least partially structured with an elastic member, the container including inside thereof a plurality of rooms to contain solution and a passage to connect the plurality of rooms; and
a housing which is arranged with a liquid feeding device which conducts inspection of the solution by applying external force to the elastic member to move the solution in the passage or in at least one of the rooms,
wherein the housing is sealed air-tightly from outside air, and the housing is a safety cabinet to which a sterilization filter is provided at an outlet passage thereby that communicates from inside to external of the housing.

2. The inspection apparatus as claimed in claim 1, wherein
the cartridge and the liquid feeding device are arranged inside the housing, a partition wall is provided inside the housing, the partition wall partitioning a processing area in which the inspection of the solution is conduced by applying external force to the elastic member, from a containing area in which a driving source to drive the liquid feeding device is accommodated,
the liquid feeding device and the driving source are connected with each other by a transfer section which penetrates through the partition wall, and
the transfer section transfers a driving force from the driving source to the liquid feeding device, while the transfer section is sealed air-tight with respect to the partition wall.

3. The inspection apparatus as claimed in claim 2, wherein the housing is provided with a cleaning door to conduct cleaning of inside the processing area.

4. The inspection apparatus as claimed in claim 1, further comprising:
a discharge duct to emit discharge air from inside the housing; wherein the discharge duct is provided with a connecting section that connects with an external processing device.
